(19) United States
(12) Patent Application Publication
(10) Pub. No.: US 2017/0240310 A1
(43) Pub. Date: Aug. 24, 2017

(54) ASEPTIC FILLING DEVICE AND METHOD

(71) Applicant: PIERRE FABRE DERMO-COSMETIQUE, BOULOGNE BILLANCOURT (FR)

(72) Inventors: BERTRAND CORMARY, ALBI (FR); LAURENT SERRA, LA TOUR SUR ORB (FR)

(21) Appl. No.: 15/310,093

(22) PCT Filed: May 11, 2015
(86) PCT No.: PCT/EP2015/060394

§ 371 Date: Nov. 9, 2016

(30) Foreign Application Priority Data
May 9, 2014 (FR) 1454174

(51) Int. Cl. B65B 55/02; B65B 3/00; B65B 3/16; B65B 55/18; B65B 31/02; B65B 39/00; B65B 51/22; A61L 2/07; B65B 7/14

(52) U.S. Cl. CPC B65B 55/027; A61L 2/07; B65B 3/003; B65B 3/16; B65B 7/14; B65B 31/02; B65B 39/00; B65B 51/22; B65B 55/18

(57) ABSTRACT

A device to package a sterile bulk product, particularly, a dermocosmetic or galenic preparation, into a container. A sterile enclosure of the device includes a hopper containing the bulk product. The hopper includes in an axial direction, a cylindrical reservoir part and a pouring part connected to the cylindrical part. A transfer device transports the container under the hopper to fill the container in a filling zone. A blower provides ultra-filtered air from the ceiling to the floor of the enclosure, to produce a protective flow of air around the filling zone. The axis of the pouring part with an asymmetrical conical shape is not in line with the axis of the reservoir part such that the pouring opening is shifted towards the outer wall of the reservoir.

The invention relates to a method and a device for aseptic filling.

The invention is particularly but not exclusively suitable for packaging dermocosmetic or galenic products, particularly for packaging sterile cosmetic products.

More precisely, the device and method according to the invention apply to the automated industrial packaging of a sterile bulk product into sterile individual containers such as ampules, vials, syringes or tubes, without that list being exhaustive. The packaged product takes the form of a liquid, gel or cream with viscosity generally ranging from 600 cP to 45,000 cP (6 to 45 Pa.s). The term ‘sterile packaging’ thus refers to filling a sterile container with sterile product, so that the product packaged in that manner achieves a given degree of sterility with no sterilization after packaging.

In one example of the background of the implementation of the invention, the invention is used to package a sterile dermocosmetic product produced in large quantities using a device and method such as those described in document WO 2013 007755.

According to the prior art, sterile dermocosmetic products without biostatic formulations are packaged in containers with a small capacity in order to limit the risks of retrograde contamination of the product after the container has been opened; thus, once the container is open, the product must be used very rapidly. Such packaging in small single-dose quantities with a short shelf life after opening limits the possibilities of commercial development of these products. Such single-dose packaging in sterile conditions is generally carried out using a method known as the Blow-Fill-Seal technique, which does not make it possible to incorporate a sophisticated device for stoppering the container and is thus limited to single-dose packaging.

These production methods of the prior art suffer from the drawback of coupling the production of containers and the production of sterile product. For example, storing the product implies identically storing the container and vice-versa. The containers and the product must be produced at the same speed, which makes the management of production flows more complex when the product is put on sale in different possible packages.

The use of a system for closing the container hermetically as described in document FR 2 873 358 makes it possible to eliminate the risk of retrograde contamination of the product and opens the way for more customary packages, such as tubes with several hundreds of milliliters. But while packaging systems suitable for non-sterile preparations can be found for such containers, there is no sterile packaging line in the area of cosmetic or galenic products suitable for handling a production flow compatible with a sterilization method such as that described in document WO 2013 007755.

In alternative embodiments of the prior art, the product is sterilized after it is packaged, particularly by subjecting said container containing the product to ionizing radiation. That embodiment limits the choice of the type of container, particularly the material of the container, its shape, which determines the thickness of the product subjected to ionizing radiation and the inks used for decorating the container, without the list being exhaustive.

According to the prior art, applied to non-sterile packaging, the bulk preparation is placed in a hopper and a transfer device brings the container under said hopper to fill the container. Throughout the text, the word ‘hopper’ refers to a container comprising a part, known as the pouring part, that is shaped like a cone or an upside-down pyramid and acts as a funnel to direct the contents of the container towards a pouring opening, where said container is cylindrical, circular in section, rectangular or of any other shape.

According to the prior art, applied to the packaging of sterilized food products,

in order to protect the bulk product delivered from the hopper from contamination as it fills the container, an ultra-filtered flow of air is blown around the filling zone. That flow of air prevents particles from reaching the product poured into the container.

Document WO 2012 171958 describes a sterile packaging system for food products. In that installation, the filling zone, which is at a distance from the hopper, is located in a sterile enclosure protected by a laminar air flow from the ceiling to the ground. Those industrial systems of the prior art, which are suitable for the production of large volumes and essentially designed for food products, and which continuously handle the same materials, do not achieve the performance levels required for bulk protection during filling when they are used for applications in the field of galenic or dermocosmetic products. Indeed, in the area of sterile galenic or cosmetic products, the sterility requirements are much higher than in food products.

Thus, according to standard EN 556 relating to medical devices and the European pharmacopeia, a product is known as sterile when its sterilization process can reach a value F0 for 15 minutes, that is to say the equivalent of exposure to temperature of 121° C. for 15 minutes. For sterile cosmetics without preservatives, the sterility rate required corresponds to a likelihood of the proliferation of micro-organisms in the product below 10₋₆. That sterilization level in respect of sporulated geobacillus stearothermophilus is achieved by means of a heat sterilization process corresponding to F0 for 22 minutes. As a comparison, sterilization at ultra-high temperature of a food product such as milk corresponds to F0 for about 3 minutes.

That level of sterility must be retained up to final packaging. Also, the turbulence generated in the protective air flow in the filling zone of the food product according to the device of the prior art would be sufficient to raise to an unacceptable level the likelihood of the contamination of the bulk product in an application in the field of dermocosmetic products. That is because even though the environment is kept clean and sterile, in view of the volume of an industrial system, there is always a risk of a particle being introduced in the container or in the preparation at the time of filling. Paradoxically, the protective air flow becomes the main reason for the contamination of said bulk product due to the turbulence. Further, production in the area of dermocosmetic products is organized in batches of products with physical characteristics that are highly variable, particularly regarding their viscosity and individual packaging volumes. Thus, the ability to rapidly change production batches on the same line is essential.

In the solutions of the prior art for sterile packaging, such as that described in document EP 2 179 925, the hopper is distant from the filling zone. This solution of the prior art entails the presence of a length of piping between the hopper and the filling zone. But all such piping must be perfectly cleaned with each change of batch, that is to say with each change in the type of product packaged. Such cleaning is particularly difficult when the packaged product is a viscous dermocosmetic product in the form of a cream or a balm. Further, all these lengths of piping comprise zones where micro-organisms can develop in the trapped traces of product.

The invention aims to remedy the drawbacks of the prior art and therefore relates to a device for packaging sterile bulk material, particularly a dermocosmetic or galenic preparation, into a container, which device comprises:

a. a sterile enclosure, and in that sterile enclosure;

b. a hopper containing the bulk product and comprising, in an axial direction, a cylindrical reservoir part and a pouring part connected to the cylindrical part;

c. a transfer device suitable for transporting the container under the hopper in order to fill the container in a zone known as the filling zone;

d. a device for blowing ultra-filtered air from the ceiling to the floor of the enclosure, suitable for producing a flow of air known as a protective flow, around the filling zone;

e. wherein the axis of the pouring part with an asymmetrical conical shape is not in line with the axis of the reservoir part so that the pouring opening is shifted towards the outer wall of the reservoir.

Thus, even though the hopper is placed as close as possible to the filling zone, thus minimizing the length of piping between the bulk product and the containers. The presence of this hopper does not disturb the protective laminar air flow in the filling zone, thus avoiding turbulence and eliminating the risk of contaminating the product during the filling process with particles carried by said air flow.

The invention can be implemented advantageously in the embodiments described below, which may be considered individually or in any technically operative combination.

Advantageously, the device according to the invention comprises:

f. extraction means suitable for creating an air flow that scavenges the transfer device.

Those means are favorable to the achievement of a protective laminar air flow around the filling zone, and direct all particles that could contaminate the product towards extraction means from where they are removed outside the filling zone.

In a particular embodiment, the device according to the invention is suitable for filling a flexible tube closed at one end, and comprises, in this embodiment:

g. means for maintaining the flexible tube on the transfer device and holding the open end of said tube under the pouring opening;

h. a station for sealing the open end of the tube, that is supplied by the transfer device after the filling zone, wherein said sealing station is under the protective flow.

Thus, the tubes are sealed in sterile conditions just after they are filled and the product poured in the tube is only exposed to the air for a very short time and always under a protective flow of ultra-filtered air.

Advantageously, the tube is made of thermoplastic material and

crimping is carried out by welding together the edges of the open end of the tube.

This embodiment is particularly cost-effective and fast and leads to the fully sealed closing of the open end of the tube.

Advantageously, the initially closed end of the flexible tube is a cap suitable for avoiding retrograde contamination of the product contained in said tube. Thus, the product is advantageously packaged in containers adapted for regular use.

Advantageously, the means for holding the flexible tube on the transfer device comprises a cellular collector in which the flexible tubes are inserted.

Thus, each batch of tubes is supplied sterile, mounted in its collector, which is installed on the transfer device of the device according to the invention for packaging the product.

The invention also relates to a method for manufacturing and packaging a sterile bulk product, particularly a dermocosmetic or galenic preparation, using a packaging device according to the invention and a device for steam infusion sterilization comprising a steam generator, which process comprises the steps of:

i. sterilizing the bulk product in a device that uses a steam infusion method;

ii. transferring the bulk product thus sterilized to the hopper of a packaging device according to the invention;

iii. supplying a sterile assembly comprising containers held in a cellular collector;

iv. packaging the bulk product in the containers using a filling method comprising passing said containers in their cellular collector under the hopper of said packaging device under an ultra-filtered laminar flow.

This process makes it possible to make sterile dermocosmetic or galenic preparations from unsterilized raw materials without sterilizing them after packaging and thus offers great flexibility in terms of the choice of raw materials and packaging, regarding both the volume and the type of packaging (tube, ampule), and the material of which said packaging is made.

In one alternative embodiment of the method, step (ii) is carried out by direct transfer from the steam infusion device to the hopper. This alternative embodiment is suitable for just-in-time production in the same geographical location. The hopper acts as the buffer volume.

In another alternative embodiment, the method according to the invention comprises, between step (i) and step (ii), a step of:

v. storing the sterilized bulk material in a closed sterile tank, wherein step (ii) is carried out by transferring the bulk product from said tank into the hopper.

This alternative embodiment makes it possible to sterilize the bulk product on one site and package it on another. More generally, it offers greater flexibility in production management.

In one particularly advantageous embodiment, the method according to the invention comprises, prior to step (i), a step of:

vi. sterilizing the whole system by circulating throughout said system high- temperature steam produced by the steam infusion means.

Thus, the steam produced by the device used for production can be used to sterilize the whole system without adding a separate device. The installation is thus adapted to a rapid change in runs.

The method according to the invention is advantageously implemented with a container in the form of a tube with a capacity ranging between 50 ml and 400 ml comprising a cap that prevents retrograde contamination.

In this embodiment, the method according to the invention advantageously comprises a step of:

viii. closing the tube with a weld line after it is filled. Thus, the tube is closed hermetically before it leaves the sterile packaging zone.

Advantageously, the bulk product contained in the tube after step (viii) is so sterile that the likelihood of the development of micro-organisms in the composition is below 10₋₆. Thus, the product contained in the tube is suitable for preservation.

The invention is described below in its preferred embodiments, which are not imitative in any way, and by reference to FIGS. 1 to 5, wherein:

FIG. 1 of the prior art is a schematic representation of an exemplary embodiment of a sterile packaging device, adapted, for example, for packaging a food product;

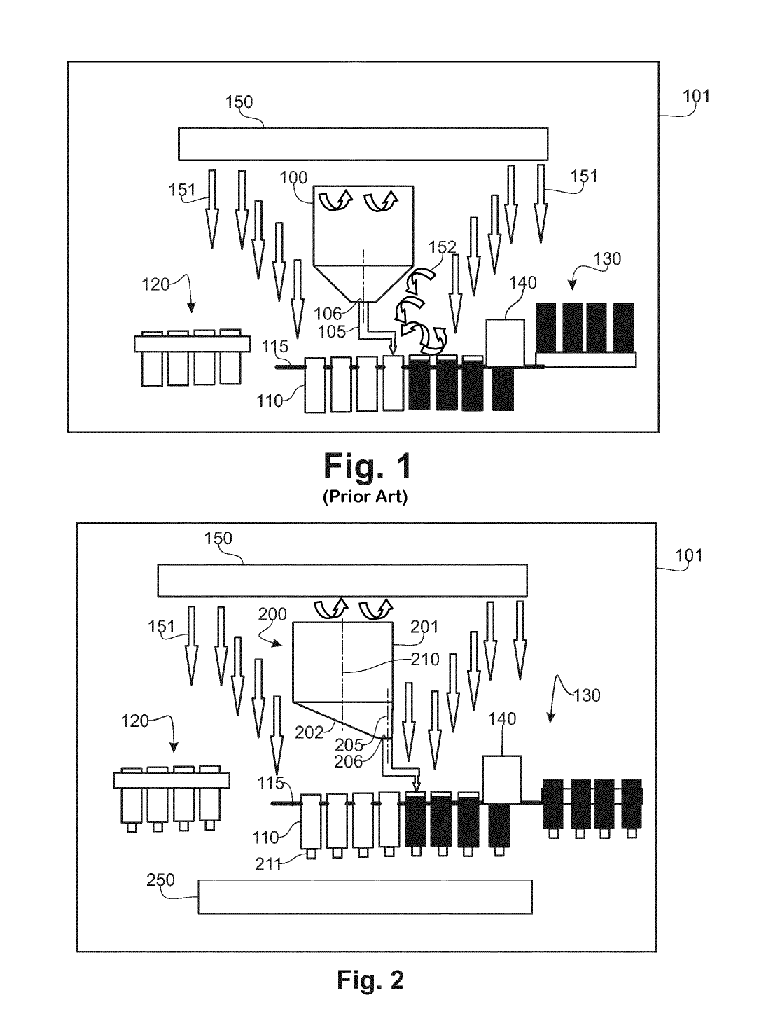

FIG. 2 is a schematic representation of an exemplary embodiment of a device for packaging a sterile bulk product according to the invention;

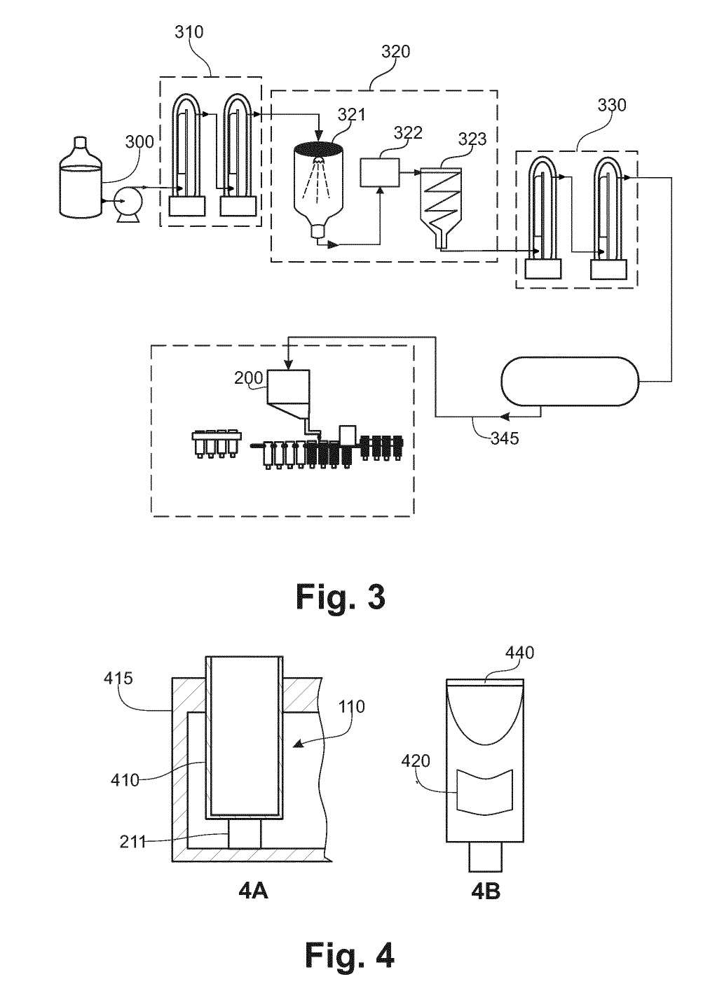

FIG. 3 is a schematic illustration of an exemplary embodiment of a production and packaging system for sterile dermocosmetic product;

FIG. 4 is an exemplary embodiment of a container for a sterile dermocosmetic preparation according to the invention, FIG. 4A is a sectional front view before said container is filled, held in a cellular collector, and FIG. 4B is a front view after filling and crimping;

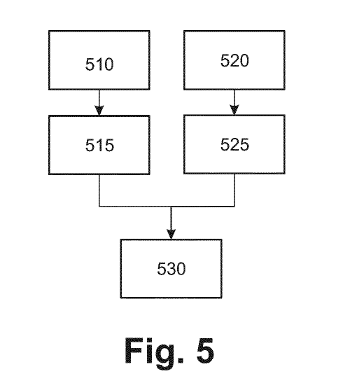

and FIG. 5 is a logical diagram of the method for packaging sterile bulk product according to the invention.

In FIG. 1 of an exemplary embodiment of the prior art applied particularly in the case of the filling of a container (110) with a food product, the bulk product is contained in a hopper (100). The product is poured from the hopper (100) into the container (110) through a filling nozzle connected to the pouring opening (106) of the hopper (100).

The containers (110) are carried by a transfer device (115) which takes them from a loading station (120) to an unloading station (130). The whole machine is placed in an enclosure (101) with access through an air lock. After the container (110) is filled, it is closed and sealed by a closing device (140), before it is sent to the unloading station (130). In order to make sure that there is no contamination by particles during the filling process, a blowing device (150) generates a flow of ultra-filtered air from the ceiling to the floor of the device. That air flow (151) is mostly laminar in the volume of the enclosure; however, the presence of the hopper (100) creates turbulence (152) in that air flow, particularly in the filling zone. That turbulence is liable to lead to the contamination of the containers by particles. If the bulk product is a food product, the risk of contamination generated by the turbulence is acceptable, as the enclosure of the machine itself is clean and sterilized.

With a cosmetic product containing no preservatives, the sterility rate required does not tolerate such a contamination risk.

In FIG. 2, in order to remedy the drawbacks of the prior art, the device according to the invention uses a special hopper (200) comprising a part (201) known as the reservoir, which is cylindrical, followed axially by a part (202) known as the pouring part, connected to the cylindrical part (201) with an asymmetrical conical shape. The axis (205) on which the top of the cone defining the shape of the pouring part (202) is located is offset in relation to the axis (210) of the cylindrical part (201). Thus, the pouring opening (206) of the hopper, which opening is substantially lined up with the axis (205) of the top of the shaping cone of the pouring part (202), is offset towards the outer wall of the reservoir part (201) of said hopper (200). That arrangement makes it possible to retain a laminar flow in the filling zone of the container and thus considerably limit the risks of contaminating the product with particles during filling. Indeed, in this arrangement, the cylindrical part (201) known as the reservoir, does not hide the filling zone from the flow (150) of air from the ceiling. The device according to the invention comprises, in this exemplary embodiment, an extraction device (250) placed under the transfer device (115). That extraction device scavenges said transfer device with an air flow and also favors the creation of a laminar air flow around the filling zone in the configuration of the device according to the invention. In this exemplary embodiment, the container (110) is a tube with an end that is open for filling while the other end is closed by a special cap (211) suitable for preventing any retrograde contamination of the product contained in the tube when said tube is used by the consumer. The enclosure of the tube (110) is made of thermoplastic material and the closure of the container is made by welding the edges of the open end.

In this exemplary embodiment, said tubes have a capacity from 50 ml to 450 ml, but these values are not limitative. In this example, the bulk product is a dermocosmetic product that takes the form of an emulsion with viscosity ranging between 600 cP and 45,000 cP.

In FIG. 3 of an exemplary embodiment of the method according to the invention, the dermocosmetic or galenic preparation (300) is heat sterilized using an UHT or ‘ultra-high temperature’ process. To that end, in this exemplary embodiment, during a pre-heating step (310), said preparation is heated to an appropriate temperature, particularly to give it viscosity adapted to the sterilization treatment intended. In this exemplary embodiment, the sterilization step (320) is carried out by steam infusion. According to this method, the pre-heated preparation is introduced in the form of a jet in an enclosure (321) comprising clean water vapor at a definite temperature. The preparation is heated instantly due to the large exchange surface between the streams of the preparation and the vapor, and the condensation of the vapor on said streams. The preparation is held (322) at that temperature for a very short time, depending on the sterilization level required, before it is injected in a vacuum chamber (323) where said preparation undergoes very rapid cooling due to the vaporizing, under the vacuum, of the water that has condensed during the heating stage (321). The preparation is then cooled (330) gradually as it is stirred to a temperature that is favorable for packaging, for example temperature ranging from 30° C. to 40° C. In this exemplary embodiment, the preparation is stored in a closed buffer tank (340). In an exemplary embodiment of the method according to the invention, said buffer tank (340) is connected by piping (345) to the hopper (200) of the packaging device according to the invention. The sterilization conditions, namely the temperature and speed of pre-heating, the temperature and time of steam infusion sterilization and the speed of cooling after sterilization are selected so that the emulsion that makes up the preparation is not broken during the process, that is to say that said preparation does not lose its emulsion properties and retains its sensory characteristics. Thus, the preparation sterilized by the steam infusion process is transferred in the form of a sterile bulk product into the packaging device without any other treatment. The bulk product is transferred into the hopper (200) through sterile piping (345) so that said bulk product is not in contact with the air, thus eliminating the risk of contamination prior to filling.

Advantageously, in this configuration of implementation of the method according to the invention, the steam generator of the steam infusion sterilization device (320) is used to sterilize the entire system, by circulating steam in the entire production and packaging circuit, that is to say particularly the piping, the heating and cooling devices, the buffer tank (340) and the hopper (200), when said circuit is entirely drained of product. Alternatively, the sterile preparation is stored in the sealed buffer tank (340) and taken to a place of packaging that is distant from the place of sterilization.

In FIG. 4A, prior to the packaging operation, the sterile empty tubes (110) are placed in a cellular collector (415) which may for instance be made of cellular polypropylene plates. The special caps (211) that prevent retrograde contamination are installed at the ends of said tubes (110). The whole is sterilized and bagged. As a non-limitative example, the enclosure (410) of the tube (110) is made of single-layer polyethylene. In an alternative embodiment, the tube enclosure is made of several layers and comprises a layer of polymer that is

impermeable to oxygen. In alternative embodiments, the enclosure (410) of the tube (110) is made of metal. In practice, the choice of tube material is very wide, as the method and device according to the invention do not require sterilization after packaging.

In FIG. 4B, the filled tubes do not undergo any other sterilization operation and empty tubes are installed in the cellular collectors after the tubes are printed. Advantageously, the tube (110) and said collector (415) comprise marks (not shown) adapted to cooperate to correctly direct the printed part (420) of the tube in relation to the weld line (440) that closes the tube after it is filled.

In FIG. 5, the method according to the invention makes it possible to achieve an industrial production mode with production speeds of about 2000 kg per hour, to produce sterile dermocosmetic or galenic product that is free from preservatives, in a container that can be retailed directly and comprises all the mandatory information and appropriate distinctive signs. Said method comprises a step (510) for the production of sterile bulk product, particularly by means of a steam infusion sterilization method, and at the same time, a step (520) for supplying sterile containers with collectors, known as a sterile assembly, particularly in a bagged cellular collector.

Thus, unlike the methods of the prior art of the Blow-Fill-Seal type for single- dose packages, or sterilization of product in the containers, the method according to the invention makes it possible to separate the two production flows and thus offers greater production flexibility while retaining the advantages of just-in-time production. In a step (525) for preparing the containers, the collector is removed from its bag and placed on the table of the packaging device according to the invention, entirely in a clean environment. At the same time, in a step (515) for supplying bulk product, the sterile bulk product produced in the previous step (510) is introduced into the hopper of the filling device according to the invention. In alternatives of the method according to the invention, the bulk product thus introduced in the hopper comes directly from the sterilization device or the hopper is supplied from a sterile tank. The containers and the bulk product are transferred into the packaging device, and the containers are filled and sealed during a filling step (530).

As an example, the method and device according to the invention make it possible to produce sterile product for distribution into tubular containers with a capacity ranging from 50 ml to 400 ml. Said product takes the form of an emulsion, and the composition packaged in the container is sterile, with a probability of proliferation of micro-organisms below 10₋₆, even though it is free from preservatives. This type of product is intended to be applied on the skin, and cannot be produced in single-dose packaging.

A product that is free from preservatives means that it is free from compounds intended to prevent the proliferation of bacteria or fungi in the formula, such as in particular the products known as paraoxybenzoates or parabens, triclosan, cetrimonium bromide, methylisothiazolinone, formaldehydes, phenoxyethanol or sulfate detergents, without this list being exhaustive.

Thus, the use of the combination of the method for sterilization and packaging according to the invention makes it possible to propose such a sterile product that is free from preservatives, in packaging that allows easy use and application.

That is because the tube, with its cap that prevents retrograde contamination, makes it possible to remove a dab of product and apply it on the skin, and to repeat that operation as often as necessary to cover the entire application surface without wasting the product, and keeping it sterile at the same time.

In an exemplary embodiment, the method and device according to the invention are implemented for manufacturing and packaging a sterile cream without preservatives, comprising:
2% to 5% cetearyl glucoside;
0.1% to 0.5% carbomer;
5% to 12% glycerin;
2% to 10% caprylic capric triglyceride;
5% to 10% liquid paraffin;
water to make up 100%.

In this exemplary implementation, the product is packaged in tubes containing 50 ml to 400 ml of product and closed by a cap that prevents retrograde contamination.

Thus, the method and device according to the invention make it possible to sterilize and package products which, due to their viscosity or the presence of solid or waxy compounds, cannot be sterilized by being passed through a membrane, and the containers of which do not lend themselves to sterilization in the container.

The description above and the exemplary embodiments show that the invention achieves its objectives; in particular, it allows a flexible and just-in-time industrial production process for producing a sterile dermocosmetic or galenic preparation at high production speeds, with high sterility compliance, that is to say with F0 above 15 minutes, throughout the production line. It makes it possible to propose sterile products that are free from preservatives in containers with large volumes, that allow easy and economical application of said products.

1-13. (canceled)

14. A packaging device to package a sterile bulk product into a container, comprising:
a sterile enclosure comprising:
a hopper containing the sterile bulk product and comprising, in an axial direction, a cylindrical reservoir part and a pouring part connected to the cylindrical part;
a conveyor to transport the container under the hopper to fill the container in a filling zone;
a blower to provide ultra-filtered air from a ceiling to a floor of the sterile
enclosure, to produce a protective flow of air around the filling zone; and
wherein an axis of the pouring part with an asymmetrical conical shape is not in line with an axis of the cylindrical reservoir part such that a pouring opening of the pouring part is shifted towards an outer wall of the cylindrical reservoir.

15. The packaging device according to claim 14, wherein the sterile bulk product is a dermocosmetic or galenic preparation.

16. The packaging device according to claim 14, further comprising an extractor configured to create an air flow that scavenges the conveyor.

17. The packaging device according to claim 14, wherein the container is a flexible tube, closed at one end; and further comprising:
a collector to maintain the flexible tube on the conveyor and to hold the open end of the flexible tube under the pouring opening; and
a sealing station to seal the open end of the flexible tube conveyed from the filling zone by the conveyor, wherein the sealing station is under the protective flow.

18. The packaging device according to claim 17, wherein the flexible tube is made of a thermoplastic material; and wherein the sealing station crimps by welding together edges of the open end of the flexible tube.

19. The packaging device of claim 17, wherein an initially closed end of the flexible tube is a cap configured to avoid a retrograde contamination of a product contained in the flexible tube.

20. The packaging device according to claim 17, wherein the collector is a cellular collector in which the flexible tube is inserted.

21. A method for manufacturing and packaging a sterile bulk product using the packaging device according to claim 14 and a steam infusion sterilizer comprising a steam generator, the method comprising the steps of:
sterilizing a bulk product in the steam infusion sterilizer;
transferring the sterilized bulk product to the hopper of the packaging device;
supplying a sterile assembly comprising containers held in a cellular collector; and
packaging the sterilized bulk product in the containers by passing the containers in their cellular collectors under the hopper of the packaging device to fill the containers under an ultra-filtered laminar flow.

22. The method according to claim 21, wherein the sterile bulk product is a dermocosmetic or galenic preparation.

23. The method according to claim 21, wherein the transferring step is carried out by a direct transfer from the steam infusion sterilizer to the hopper of the packaging device.

24. The method according to claim 21, further comprising, between the sterilizing and transferring steps, a step of storing the sterilized bulk product in a closed sterile tank; and wherein the transferring step is carried out by transferring the sterilized bulk product from the closed sterile tank into the hopper of the packaging device.

25. The method according to claim 21, further comprising, prior to the sterilizing step, a step of sterilizing a whole system used to manufacture and package the sterile bulk product by circulating throughout the system a high-temperature steam produced by the steam infusion sterilizer.

26. The method according to claim 21, wherein the container is a tube with a capacity ranging between 50 ml and 400 ml, the tube comprises a cap to prevent a retrograde contamination.

27. The method according to claim 26, further comprising a step of closing the tube with a weld line after it is filled.

28. The method according to claim 27, wherein the sterile bulk product comprises the following material to exclusion of any preservative:
2% to 5% cetearyl glucoside;
0.1% to 0.5% carbomer;
5% to 12% glycerin;
2% to 10% caprylic capric triglyceride;
5% to 10% liquid paraffin; and
water to make up 100%.
